(19) United States
(12) Patent Application Publication
(10) Pub. No.: US 2013/0022666 A1
(43) Pub. Date: Jan. 24, 2013

(54) METHODS AND COMPOSITIONS FOR TRANSFER OF MITOCHONDRIA INTO MAMMALIAN CELLS

(71) Applicant: Anna Brzezinska, Wauwatosa, WI (US)

(72) Inventor: Anna Brzezinska, Wauwatosa, WI (US)

(21) Appl. No.: 13/547,102

(22) Filed: Jul. 12, 2012

Related U.S. Application Data
(60) Provisional application No. 61/509,903, filed on Jul. 20, 2011.

(51) Int. Cl. A61K 35/12; A61K 9/133; A61K 9/127; A61P 9/00; C12N 5/071

(52) U.S. Cl. 424/450; 424/569; 435/375

(57) ABSTRACT

Disclosed are compositions comprising a lipid carrier and a mitochondria. Also disclosed are methods of delivering exogenous mitochondria to a cell and methods of treating or reversing progression of a disorder associated mitochondrial dysfunction in a mammalian subject in need thereof

### CROSS-REFERENCE TO RELATED APPLICATIONS

This application claims the benefit of priority to U.S. Provisional Application No. 61/509,903, filed Jul. 20, 2011, which is incorporated by reference in its entirety.

### STATEMENT REGARDING FEDERALLY SPONSORED RESEARCH

This invention was made with support from the National Institute of Health GM: P01GM066730, Project 1. The United States Government has certain rights in this invention.

### INTRODUCTION

The primary function of mitochondria is to provide energy for cellular metabolic processes. Moreover, mitochondria function as sensors and regulators of metabolic and signaling pathways within the cell cytoplasm (1-3). Therefore, mitochondrial dysfunction can affect physiological performance of cells at different levels. The intracellular location of mitochondria restricts access to the organelle, thereby greatly diminishing the potential for extracellular medical intervention. Mitochondrial dysfunction, as assessed by a decrease in energy production, induces a complex phenomenon that involves changes in the products of mitochondrial metabolism. These changes include increased generation of superoxide, and hence increased oxidative stress (4), an increased rate of mitochondrial DNA mutations (5), and impaired function of the elements of oxidative phosphorylation (6). Changes in cellular metabolism may cause modifications of mitochondrial components such as oxidation or nitrosylation of proteins located on mitochondrial membranes or in the mitochondrial matrix (7). Thus, mitochondria are both the source of and the target of induced metabolic changes, and those changes in mitochondrial metabolism that result in decreased energy production are largely irreversible. Cells control the number of dysfunctional mitochondria by way of mitophagy (8). However, if the mitochondrial dysfunction exceeds the ability of cells to correct these errors, mitochondrial dysfunction will permanently affect the bioenergetic and metabolic profiles, which can contribute to development and progression of disease processes.

Clinical symptoms of mitochondrial dysfunction can only be partially ameliorated by medical treatments. However, the immediate source of mitochondrial dysfunction, mitochondria, have not been targeted effectively for direct treatment. One approach to reversing mitochondrial dysfunction that has been reported involved transferring the intact non-human spindle chromosomal complex into enucleated oocytes of an unrelated female primate, which allowed production of non-human primate offspring that did not suffer from mitochondrial disease (9). In another approach, polynuclear transfer between human zygotes led to the development of embryos with normal mitochondria (10). A third approach used the formation of hybrid cells by merging enucleated donor cells with cytoplasm containing mitochondria with arget cells in which mitochondrial DNA had been experimentally depleted (10-12). Other attempts to reverse mitochondrial dysfunction include direct in vitro delivery of drugs contained within liposomes to mitochondria (13, 14), and establishing cell-to-cell connections by way of microtubules to transfer mitochondria between mother and daughter cells (15). Finally, the direct absorption of exogenous mitochondrial protein through the plasma membrane has also been attempted (16, 17). All approaches mentioned are of limited utility, and treatments targeted to the whole organism are associated with adverse effects or ethical challenges. Thus, it is necessary to access mitochondria directly and modify their function in a way that will allow application both in vitro and in vivo.

### SUMMARY

In certain embodiments, the present invention provides a composition that includes a lipid carrier within which is contained one or more mitochondria. Examples of suitable lipid carriers include liposomes, lipid microtubules, lipid microbubbles, and lipid microspheres. Advantageously, these compositions may be used to deliver mitochondria to cells. The mitochondria may be from an autologous or heterologous source. In certain embodiments, targeting to certain cell types may be enhanced by using compositions comprising a targeting moiety that targets the lipid carrier to certain types of cells or tissues. Examples of suitable targeting moieties include antibodies and receptor ligands.

Certain embodiments provide a method for delivering exogenous mitochondria to a cell with reduced or defective mitochondria. The method involves contacting the cell with the composition of the invention under conditions that allow uptake of the mitochondria by the cell. In certain embodiments, the method is accomplished by administering the composition of the invention to a mammal having a disorder characterized by deficient or defective mitochondria. The composition may be administered by any suitable method, including, for example, orally, intraperitoneally, intravenously, subcutaneously, intramuscularly, or by inhalation.

The present invention also provides a cell comprising exogenous mitochondria.

In certain embodiments are provided a method of treating and/or reversing progression of a disorder associated mitochondrial dysfunction by administering the composition of the invention to a mammal with the disorder.

### BRIEF DESCRIPTION OF THE DRAWINGS

FIG. 1. Characterization of endothelial cells. (A) Mitochondrial networks in NT (A), HT (B) and NT-p° (C.) cells. (D) Intracellular ATP levels in NT (N=3, n=59), HT (N=5, n=81), and NT-p° cells (N=5, n=70), respectively. Results are mean ±SD, * P<0.05.

FIG. 2. Uptake of exogenous mitochondria by recipient HT cells. (A) Levels of autofluorescence in HT cells. (B) Delivered mitochondria tagged with NOA and visualized in the recipient HT cells. (C) TMRE (only in red) showing endogenous mitochondria, and an overlay with (B) in orange (TMRE +NOA). (D) Endogenous mitochondria marked with TMRE. (E) Exogenous mitochondria tagged with Qdots (blue). (F) Overlay of (D) and (E) exogenous mitochondria (pink) in the recipient HT cells. All images were recorded 24 hours after treatment of the recipient cells with mitochondria-liposome complex.

FIG. 3. Intracellular ATP in the recipient HT cells. (A) Intracellular ATP measured 24 hours after HT cells were treated with the mitochondria-liposome complex. (N=3, n=25-50). (B) A sustained increase in intracellular ATP was measured 72 hours after transfer (N=5, n=85-120). Results are mean ±SD, * P<0.05.

FIG. 4. Uptake of exogenous mitochondria by recipient NT-p° cells. (A) TMRE fluorescence in control cells. (B) NOA-tagged exogenous mitochondria. (C) Overlay of (A) and (B) shows exogenous mitochondria in orange (TMRE+NOA) and endogenous mitochondria in red (only TMRE). (D) Magnification represents an overlay with exogenous (green, red, and orange) and endogenous (red) mitochondria. Images were recorded 24 hours after treatment of the recipient cells with the mitochondria-liposome complex. (E) Intracellular ATP levels in NT-p° cells 24 hours after treatment with (N=5, n=70-100) and (F) 96 hours after treatment with mitochondria-liposome complex (N=5, n=69-100). Results are mean ±SD, * P<0.05.

FIG. 5. Effect of mitochondria-liposome complex on proliferation of HT and NT-p° cells. (A) Incorporation of BrdU into DNA in HT cells 72 hours after treatment with the mitochondria-liposome complex (N=3, n=28-50). (B) Incorporation of BrdU into DNA in NT-p° cells 96 hours after treatment with the mitochondria-liposome complex (N=3, n=19-35). Results are mean ±SEM, * P<0.05.

FIG. 6. Effect of denatured mitochondria on intracellular ATP in HT cells. Mitochondria were isolated from NT cells, heat inactivated (95° C., 120 s), and enclosed in liposomes. HT cells were exposed to mitochondria-liposome complex for 3 hours. Intracellular ATP was measured 12 hours after the treatment with the mitochondria-liposome complex (N=3, n=15-34). Results are mean ±SD, * P<0.05.

FIG. 7. Recovery of nitric oxide production in endothelial cells from hypertensive donors treated with mitochondria-liposome complex. Briefly, human coronary artery endothelial cells from hypertensive donors were divided into three groups: controls, cells exposed to mitochondria-liposome complexes, or cells exposed to empty liposomes. After one hour, media with liposomes was removed, cells were washed with HBSS, fresh cell culture medium was applied to the cells and the cells were held in the the cell culture incubator for 24 hrs, along with plates containing culture media only (no cells). After 24 hrs., 500 μl samples were collected and used to determine the amount of nitric oxide released from the control and treated cells. Measurements were corrected by subtracting the background (media alone). Results are mean ±SD, * P<0.05.

### DETAILED DESCRIPTION

As described herein below, functional isolated donor mitochondria enclosed within lipid carriers such as liposomes can be delivered directly to recipient cells in need of functional mitochondria, i.e., cells deficient in or containing defective mitochondria. It is envisioned that transfer of mitochondria will be effective in treating and/or reversing progression of disorders associated mitochondrial dysfunction.

As described in the Examples below, functional donor mitochondria isolated from normotensive human coronary artery endothelial cells were enclosed within liposomes and delivered to energy-impaired recipient endothelial cells, including endothelial cells from hypertensive donors (HT cells) and endothelial cells with depleted mitochondrial DNA (NT-p° cells). Following mitochondrial transfer, both HT and NT-p° recipient cells recovered the ability to produce energy and regained metabolic functions. Thus, transfer of exogenous mitochondria can be used to reverse progression of mitochondrial dysfunction in vascular endothelial cells.

In the Examples, liposomes prepared from soybean phosphatidylcholine were used. However, it is envisioned that any suitable lipid may be used in the lipid carriers and methods of the invention, including, but not limited to, L-α-phosphatidylcholine, dipalmitoleoyl L-α-phosphatidylcholine, β-arachidonoyl γ-palmitoyl L-α-phosphatidylcholine, phosphatidyl glycerol, phosphatidyl inositol, phosphatidylserine, phosphatidic acid, or cardiolipin phosphatidylglycerol, phosphatidylserine, sphingomyelin dicetylphosphate, phosphatidylethanolamine, cholesterol, and PEG-lipids. As one of ordinary skill in the art will appreciate, the lipid employed in the lipid carrier may be obtained from any suitable source.

As one skilled in the art will appreciate, liposomes may be prepared using any method suitable to produce liposomes that encapsulate mitochondria, including the method described herein. Particularly useful are methods that produce relatively large liposomes, including, for example, methods of producing giant unilamellar vesicles (28).

In addition to liposomes, suitable lipid carriers include, without limitation, oily suspensions, lipid microtubules, lipid microbubbles, and lipid microspheres (29).

The mitochondria-containing lipid carriers may be delivered by any suitable mode of administration, including, for example, orally, intramuscularly, intravenously, intraperitoneally, or by inhalation.

In addition to delivering mitochondria to endothelial cells, it is reasonably expected that the compositions and methods of the invention may be used to deliver mitochondria to any cell type. Further, it is envisioned that the compositions and methods of the invention may be used to target delivery to particular cell types by modifying the lipid carriers to include a targeting moiety. For example, as one of ordinary skill in the art will appreciate, the carriers could incorporate antibodies that preferentially bind to a cell surface antigen on the target cell of interest, or receptor ligands or portions thereof that interact with cell surface receptors on the target cell of interest.

It is envisioned that the methods of the invention may be used to treat any condition associated with mitochondrial deficiency or dysfunction. Conditions associated with dysfunctional mitochondria that may be treated with the method of the invention include those conditions that are an inherited genetic disease caused by single partial deletions or partial duplications, present as sporadic heteroplasmic large-scale rearrangements of mtDNA. Examples of conditions that may be treated using the methods of the invention include mutations that lead to the impairment of the metabolic function of mitochondria. Mutations in mtDNA may result from the progression of chronic disease processes, such as cardiovascular disease and cerebrovascular disease, including artheriosclerosis, hypertension, diabetes, coronary artery disease, congestive heart failure, myocardial infarction, peripheral vascular disease, or progression of physiological processes such as aging. Such conditions affect scores of millions of adults.

Evidence that exogenous biochemically active mitochondria can be delivered to recipient endothelial cells using a liposomal delivery system resulting in energetic recovery of the recipient cells is presented in the Examples below. Transfer of biochemically active mitochondria to recipient cells was discovered to promote recovery of physiological function and increased cell proliferation. Thus, delivered functional exogenous mitochondria either contribute to mitochondrial metabolism (in the case of HT cells) or override the existing deficient metabolism of the endogenous dysfunctional mitochondria (NT-p° cells).

Others have attempted to change mitochondrial distribution by introducing exogenous mitochondria into living cells with the hope of eliminating dysfunctional mitochondria (15, 17, 24). While such approaches were successful, they were extremely challenging and had limited potential for clinical applications. They include a repopulation of cells with exogenous mitochondria via the process of complementation and formation of cellular hybrids (12), or transfer of chromosomal material between cells (10). Although, these methods of repopulating cells with functional mitochondria are acceptable under in vitro conditions, they are not applicable for in vivo use. In one instance, isolated mitochondria were applied directly to cells in culture and they were subsequently detected in the cell cytoplasm. Adapting this approach to an in vivo setting requires the addition of extracellular protein(s), which introduces the danger of initiating inflammatory processes and sepsis (25).

The model presented here uses mitochondria and lipids, and the lipid component provides some control of the extra- and intra-liposomal environment necessary for delivery of the mitochondria to the target cells. In addition, the lipid layer provides a barrier to prevent interactions of exogenous and endogenous proteins with mitochondria. This limits the adverse effects of such interactions on endothelial cell function. Unlike a hydrid system where mtDNA depleted cells are repopulated with mitochondria and cell cytoplasm using electroporation (11, 12), the mitochondria-liposome delivery system offers a way to transport components across the plasma membrane without major modifications to either the plasma membrane or mitochondria. Indeed, donor mitochondria tagged with 10-nonyl acridine orange or quantum dots were detected within the cytoplasm of the recipient HT and NT-p° cells. Therefore, mitochondria were transferred from the extracellular space and internalized by the endothelial cells. Notably, NAO and Qdots fluorescence was present in HT and NT-p° cells 24 hours after exposure of recipient cells to the mitochondria-liposome complex, which is evidence that the exogenous mitochondria were retained within the cytoplasm of the recipient endothelial cells. Neither Qdots nor NAO allow for longer-term monitoring of the fate of mitochondria delivered to the recipient cells (26). However, exogenous mitochondria delivered to HT and NT-p° cells were biochemically active as demonstrated by their ability to sequester the fluorescent probe TMRE. Thus, exogenous mitochondria delivered into recipient cells retained an electrochemical gradient across the mitochondrial membrane necessary for TMRE probe access. This confirms that the mitochondria-liposome complex delivered intact functional mitochondria to the cells and that they improved the bioenergetic profile of the cells.

Energy metabolism recovered as demonstrated by the increased levels of ATP in treated HT and NT-p° cells. This increase was sustained and remained high at 72 and 96 hours after treatment. In the HT cells, recovery allowed cells to attain the same ATP as levels those measured in control cells, suggesting that the exogenous mitochondria not only survived in the recipient cells, but also contributed to cellular energy metabolism.
The results in the NT-p° cells were also sustained over time. Under control conditions (uridine-dependent growth), mitochondrial function was suppressed because of defects in oxidative phosphorylation. However, increased ATP levels were measured at 24 and 96 hours after exposure to the mitochondria-liposome complex, a result that is consistent with successful transfer of the mitochondria from the mitochondria-liposome complex to the cells, which then survived and had increased ATP levels due to the metabolic activity of exogenous mitochondria introduced. Moreover, cells treated with mitochondria-liposome complex grew in the absence of uridine. Thus, the recovery of energy metabolism observed 96 hours after treatment with the mitochondria-liposome complex was sufficient for cellular metabolic needs, and the amounts of ATP produced were comparable to those measured in normal cells.

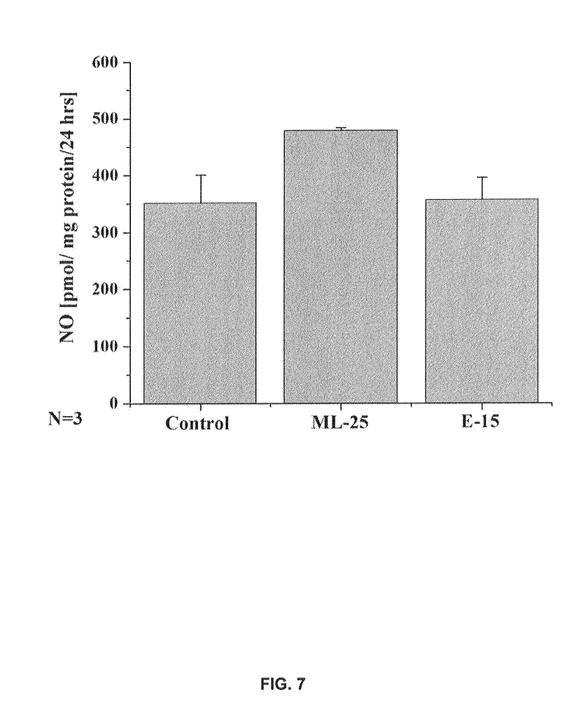

Mitochondria-liposome treated HT and NT-p° cells showed increased cell proliferation. Moreover, in the HT cells exposure of cells to mitochondria-liposome complex lead to improve release of nitric oxide (FIG. 7) by these cells. Levels of nitric oxide were increased approximately twenty percent in treated HT cells, which suggests that increased levels of ATP may have direct effects on heat shock protein 90, which is an ATP-dependent chaperone and cofactor for NO synthesis (27). Thus, transfer of the mitochondria-liposome complex into dysfunctional recipient endothelial cells improves their function. Both mechanisms, proliferation and increased nitric oxide release require improvement in the bioenergetic state of cells. Thus, the transfer of mitochondria lead to the improvement in the metabolic and energetic state of HT and NT-p° cells.

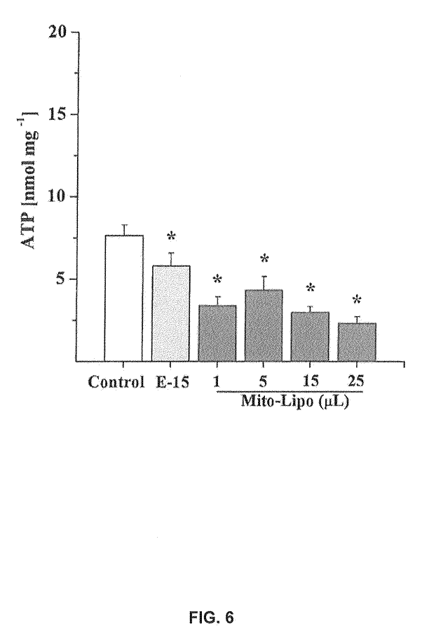

Since the recipient endothelial cells generate more ATP after treatment with mitochondria-liposome complex, the mitochondria transferred must be intact. Any change in mitochondrial integrity such as heat shock (95° C., 120 s) or physical damage (swelling, sonication, FIG. 6) before formation of the mitochondria-liposome complex has adverse effects on cell function that cause both decreased intracellular ATP levels and cell viability. Although technical challenges remain, the mitochondria-liposome complexes provide a novel and useful approach for changing the mitochondrial profile of cells with existing mitochondrial dysfunction or mitochondrial disease.

Thus, several lines of evidence support of the feasibility of direct transfer of exogenous mitochondria into endothelial cells using a liposomal delivery system. This procedure improved energy metabolism of endothelial cells obtained from hypertensive donors as well as endothelial cells that were depleted of mtDNA (NT-p°). This direct approach increases the ratio of functional to dysfunctional mitochondria within cells and improves metabolism of recipient cells. Moreover, as discussed above, such organ- and cell-targeted delivery systems could minimize or eliminate potential side effects inherent in other methods of mitochondrial transfer to cells. Thus, transfer of exogenous mitochondria to a target organ or cells may lead to subsequent repopulation of cells in which failure of mitochondrial function occurred as a result of inherited defect or progression of disease process or aging. Thus, direct transfer of exogenous functional mitochondria into cells provides a new therapeutic approach permitting changes in the bioenergetic profile of recipient cells affected with mitochondrial dysfunction, consequently leading to alleviation of defects in energy production (ATP) presented in cardiovascular diseases and in genetically inherited mitochondrial diseases.

### EXAMPLES

### Materials and Methods

Cell Culture. Human coronary artery endothelial cells from normotensive (NT) and hypertensive (HT) donors were purchased from Cell Applications (San Diego, Calif.) and sub-cultured in commercial media (Cell Applications, cat. no. 212PR-500).
Depletion of mitochondrial DNA (NT-p°). Mitochondrial DNA was depleted in endothelial cells from normotensive donors according to the method of King and Attardi (12, 18) with modifications by Yang and Loscalzo (19). Endothelial cells (NT) were treated with commercial media supplemented with 90 ng/ml ethidium bromide and 100 μg/ml uridine (NT-p°) for ten weeks. Uridine was used as a rate-limiting substrate. Depletion of mitochondrial DNA was confirmed by the absence of mitochondrial membrane potential (as assessed by confocal microscopy) and by the lack of cell growth in the absence of uridine (18).
Mitochondrial isolation. Mitochondria were isolated from endothelial cells using a commercial mitochondrial isolation kit (Thermo Scientific, Rockford, Ill.). Protein concentrations were determined using the Bradford assay (Thermo Scientific, Rockford, Ill.). Mitochondria able to consume oxygen in the presence of succinate (1 mM) were selected for subsequent use.
Liposomes. Liposomes were prepared from soybean phosphatidylcholine (PC) (Avanti Polar Lipids, Alabaster, Ala.). Lipids (50 mg/ml) were hydrated in buffer containing (in mM): 225 mannitol, 75 sucrose, 0.5 EGTA, 5 HEPES, pH 7.4 (adjusted with KOH). Lipids suspended in buffer were agitated at 220 rpm using a rotator (Lab-line Inc., Melrose Park, Ill.) at room temperature (30 min) and at 4° C. (30 min). Hydrated lipids were sonicated for 30 minutes at 4° C., to yield a hazy, transparent solution that contained liposomes (20-22).
Formation of mitochondria-liposome complexes. Mitochondria and liposomes were mixed at a 1:1 ratio (w/w) and subjected to three cycles of freezing and thawing. In the freezing cycle, the temperature of the mixture was lowered to −30° C. within 1 minute and then maintained at −80° C. for 4 minutes. In the thawing cycle, the mixtures was transferred to a 37° C. water bath for 1 min and then placed on ice for 5 minutes. Liposomes were collected by centrifugation (30 min, 14,000 rpm, 4° C.), the buffer was exchanged with hypotonic buffer (20 mM HEPES, pH 7.2, (adjusted with KOH)) the mixture was placed on ice for 5 min, and the buffer was replaced with the standard buffer (225 mM mannitol, 75 mM sucrose, 0.5 mM EGTA, 5 mM HEPES, pH 7.4 (adjusted with KOH)). This procedure was repeated two more times. Finally, liposomes or mitochondria-liposome complexes were added directly to the recipient cells. Empty liposomes were prepared in mitochondrial isolation buffer and used as negative controls for all experiments (E-15). In some experiments, mitochondria were exposed to thermal shock (95° C., 120 seconds), enclosed in the liposomes, and delivered to recipient cells or exposed to hypotonic shock, sonicated for 3 min, enclosed in the liposomes, and delivered to recipient cells as described below.
Delivery of mitochondria-liposome complexes to cells. Endothelial cells obtained from HT donors and NT-p° cells were seeded at 30,000 per well in standard cell culture media (#212PR-500), and maintained in a cell culture incubator (37° C., 5% CO₂). At 24 hours after seeding, mitochondria-liposome complexes were added directly to the cells. Cells and mitochondria-liposome complexes remained in direct contact for 1 hour and in one instance for up to 4 hours (Qdots) in a cell culture incubator (37° C., 5% CO₂). The medium containing liposomes was aspirated, cells were washed twice with Hank's balanced salt solution, and fresh cell culture medium was added. For NT-p° cells, the fresh medium did not contain ethidium bromide or uridine.
Mitochondrial membrane potential and visualization of mitochondria. Mitochondria isolated from donor endothelial cells were tagged with 100 nM of the fluorescent dye 10-nonyl acridine orange (NAO, 490/540) (Invitrogen, Eugene, Oreg.) for 30 minutes at 4° C. and used to form mitochondria-liposome complexes. To discriminate between exogenous and endogenous mitochondria in living cells, a second fluorescent probe tetramethylrhodamine ethyl ester (TMRE, 549/574, Invitrogen, Eugene, Oreg.) was used. The accumulation of TMRE within the inner mitochondrial membrane is directly related to the mitochondrial membrane potential (Δψ) and only biochemically active mitochondria can sequester the dye (23). Confocal microscopy was used to visualize fluorescent mitochondria. Images were analyzed using MetaMorph 6.1 software (Molecular Devices, Downington, Pa.).
Quantum dot (Qdot) visualization of mitochondria transferred to recipient cells. Mitochondria were tagged with Qdots according to manufacturer's instructions with one modification: biotin labeling was performed with sulfo-NHS-LC (Thermo Scientific, Rochester, Ill.). Twenty-four hours after the transfer of mitochondria, Qdots in the recipient cells were visualized using confocal microscopy.
Intracellular ATP. ATP content was analyzed using an ATP Assay Kit (Sigma, Milwaukee, Wis.). The ATP concentration was measured using a Top Count XNT microplate luminescence counter (Packard Bioscience Co, Meriden, Conn.), and normalized to the total protein content for each sample (BCA assay, Thermo Scientific).
HT or NT-p° proliferation. Cell proliferation was measured using a BrdU assay kit with chemiluminescence detection from Exalpha Biologicals Inc. (Watertown, Mass.) according to manufacturer's instructions. Negative controls included culture media and cell cultured with no BrdU. Experiments were performed in triplicate. Data analysis. Data represent comparisons between mean values of control (non-treated cells), cells exposed to empty liposomes (E), or cells exposed to the mitochondria-liposome complex and are expressed as means ±SD. Statistical analysis was performed using Kruskal-Wallis one way analysis of variance followed by the post-hoc Dunn's test. N represents the number of independent experiments and n represents the number of independent measurements. A value of P<0.05 was considered to be statistically significant. All experiments were performed at least in triplicate.

### Results

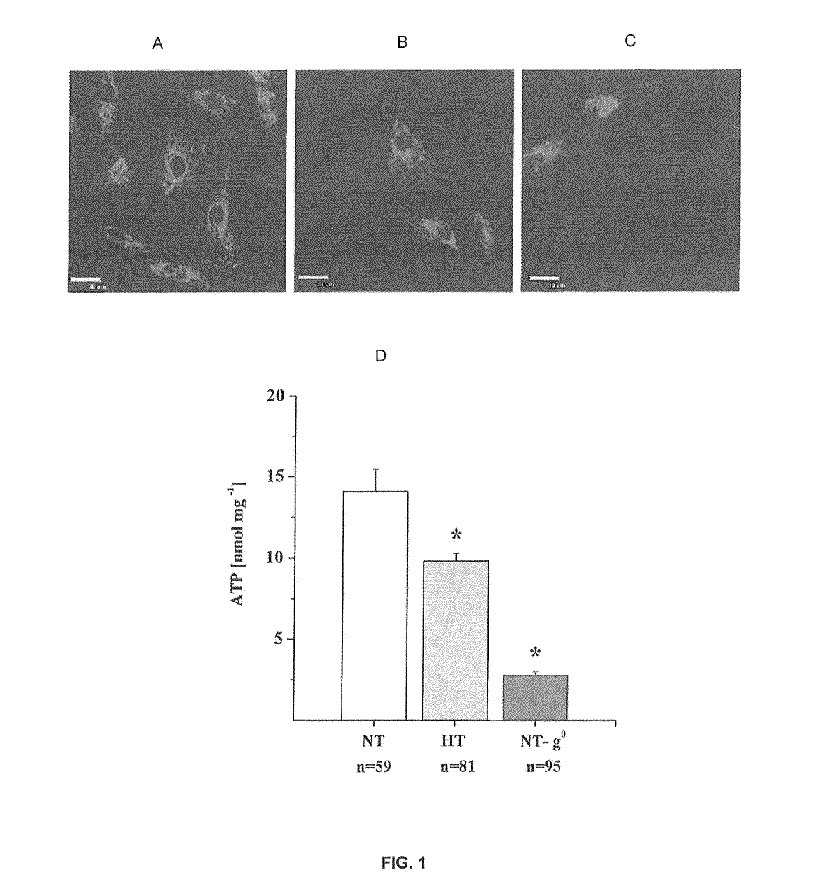

Recipient HT and NT-p° cells. Mitochondria in NT, HT, and NT-p° cells were visualized using TMRE. There were no significant differences in the distribution of mitochondria between NT and HT cells (FIG. 1 A and B). The morphology indicative of the mitochondrial network was absent in NT-p° cells (Figure C). Under control conditions, intracellular ATP was 14.1±1.4, 9.8±0.5, and 2.8±0.2 nmol/mg protein in NT cells (N=3), HT cells (N=5, P<0.05), and NT-p° (N=5, P<0.05), respectively (FIG. 1D).

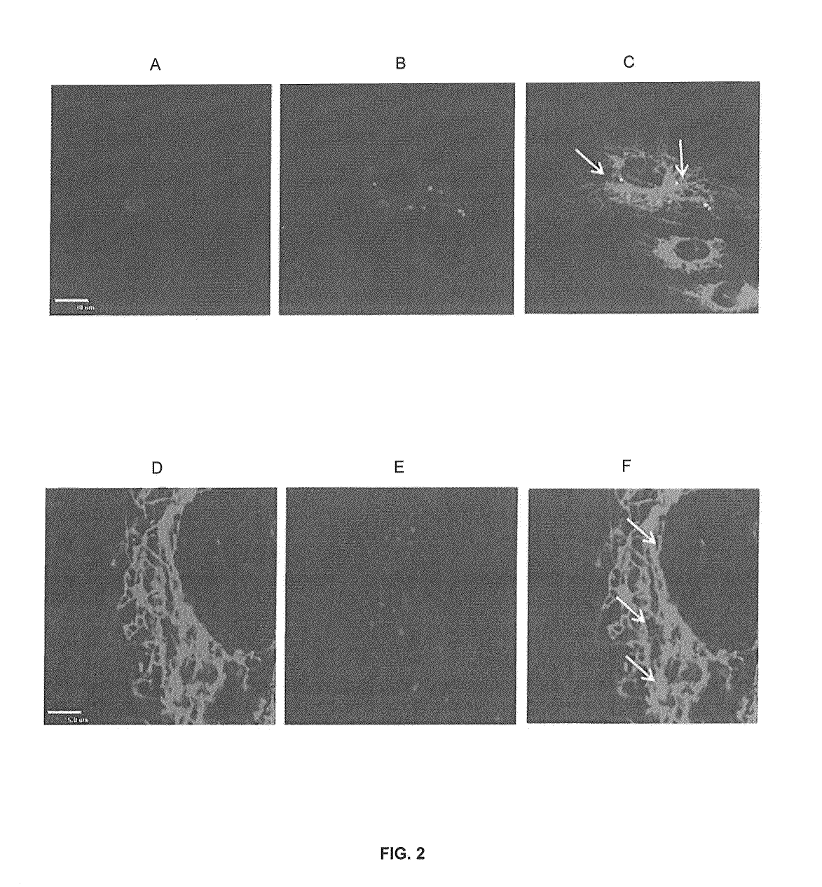

Visualization of transfer of mitochondria to recipient HT cells. To analyze successful delivery of exogenous donor mitochondria to the recipient HT and NT-p° cells, mitochondria used in the mitochondria-liposome complexes were either fluorescence-tagged with NAO (100 nM, green) or with Qdots (blue, FIG. 2) while endogenous mitochondria were unstained; therefore, fluorescence in the cell cytoplasm is from exogenous mitochondria. After exposure to the mitochondria-liposome complexes, spotted patterns of green (NAO-tagged) and blue (Qdots-tagged) mitochondria were visible in the cytoplasm of recipient HT cells. Green and blue fluorescence distributed in the cytoplasm of HT cells was detected 24 hours after treatment with mitochondria-liposome complex. To determine if the mitochondria delivered into the recipient cells were biochemically active, and retained a proton gradient across the inner mitochondrial membrane, the cells were incubated with TMRE (100 nM, 30 min). The endogenous mitochondria and most of the exogenous mitochondria delivered to the recipient cells internalized the TMRE probe (red). In NAO-tagged mitochondria (green), TMRE (red) sequestration resulted in orange fluorescence, while in Qdots-tagged mitochondria (blue) TMRE (red) sequestration yielded a pink fluorescence (FIG. 2).

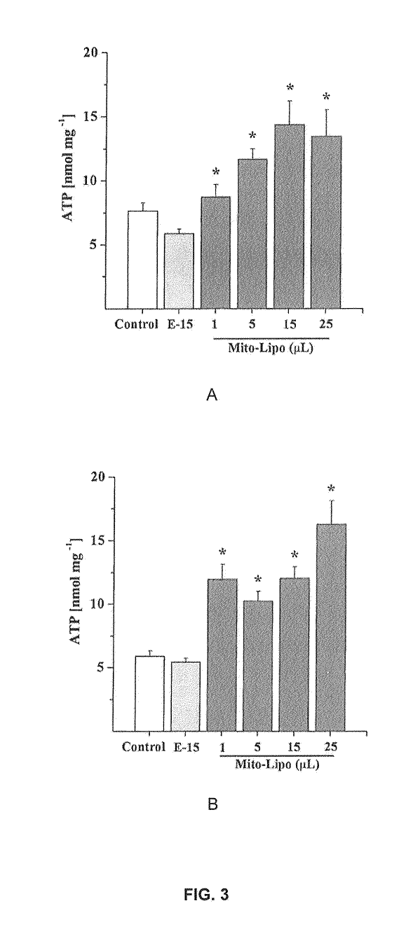

Mitochondrial transfer and intracellular ATP levels in recipient HT cells. To determine if the transfer of exogenous mitochondria increased the energy level of recipient cells, ATP was measured at 24 and 72 hours after being treated with the mitochondria-liposome complex. After 24 hours, treatment with 1-, 5-, 15- and 25-μl of the mitochondria-liposome complex resulted in an increase in the amount of intracellular ATP in recipient cells: total ATP increased from 6.2±0.3 nmol/mg protein to 8.7±0.3, 11.7±0.9, 14.4±1.8, and 13.5±2.1 nmol/mg protein (N=3, P<0.05), respectively (FIG. 3A). After 72 hours, ATP levels were 11.9±1,2, 10.3±0.7, 12.0±0.9, and 16.3±1.8 nmol/mg protein, respectively (N=5, P<0.05 vs. control, 5.9±0.4 nmol/mg protein,). Empty liposomes had no influence upon ATP levels of the recipient HT cells at 24 or 72 hours (N=5, FIG. 3B). Thus, delivery of exogenous mitochondria to HT endothelial cells increased the amount of ATP produced in recipient cells. This could be measured at 72 hrs after treatment with mitochondria-liposome complexes suggesting that lasting changes in the metabolic state of the recipient cells were achieved.

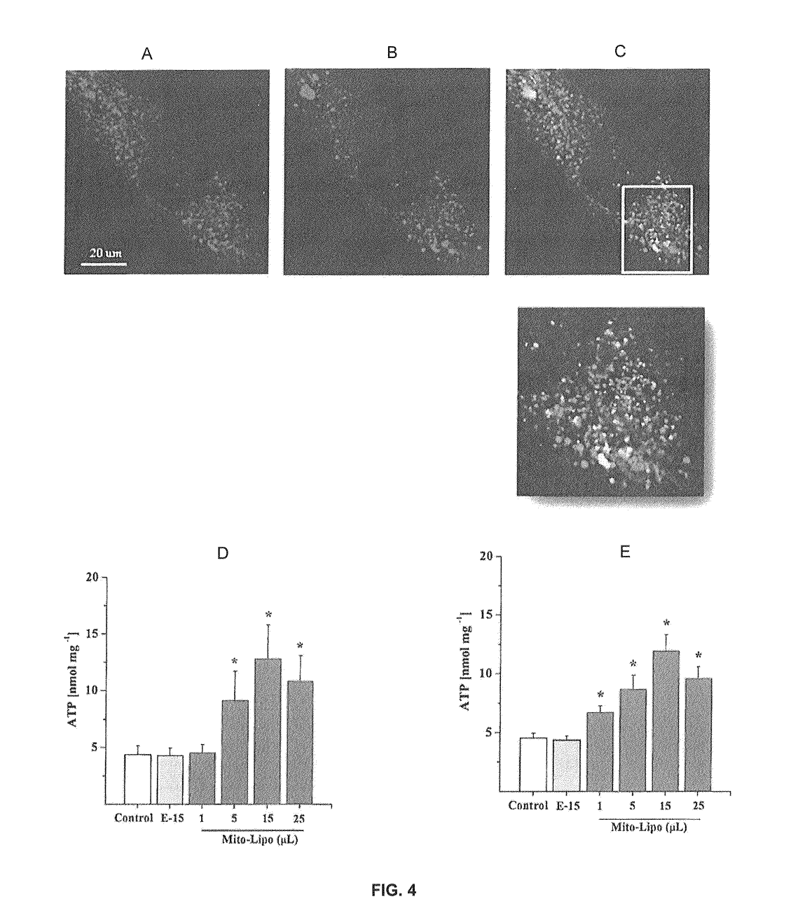

Visualization of transfer of mitochondria to recipient NT-p° cells. NAO-tagged mitochondria from NT cells were transferred to NT-p° recipient cells. The presence of NAO-tagged mitochondria was confirmed in NT-p° cells 24 hours after treatment with mitochondria-liposome complex. Mitochondria (exogenous) in NT-p° cells sequestered TMRE, and the cells remained metabolically active when uridine, a substrate of NT-p°-dependent metabolism, was removed from the cell culture media (FIG. 4A-D). Thus, the NT-p° cells treated with the mitochondria-liposome complex gained uridine-independent metabolism, which suggests normal mitochondrial function.

Mitochondrial transfer and intracellular ATP levels in recipient NT-p° cells. As compared with untreated cells, intracellular ATP in NT-p° cells was increased 24 and 96 hours after exposure to the mitochondria-liposome complex. Twenty-four hours after treatment with 1-, 5-, 15- and 25-μl of the mitochondria-liposome complex, ATP increased to 9.4±2.6, 12.8±3.0, and 10.8±2.2 nmol/mg protein, respectively (N=5, P<0.05); a lasting increase that remained for up to at least 96 hours. The amount of ATP, however, was low in untreated NT-p° cells (FIG. 4E). Thus, NT-p° cells treated with the mitochondria-liposome complex retained high levels of intracellular ATP and uridine-independent metabolism (N=5, P<0.05, FIG. 4F). The levels of intracellular ATP continued to be suppressed in NT-p° cells under control conditions and in the absence of uridine (4.5±0.4 nmol/mg protein). Empty liposomes had no effect on the ATP levels in NT-p° cells at 24 or 96 hours (FIG. 4E,F).

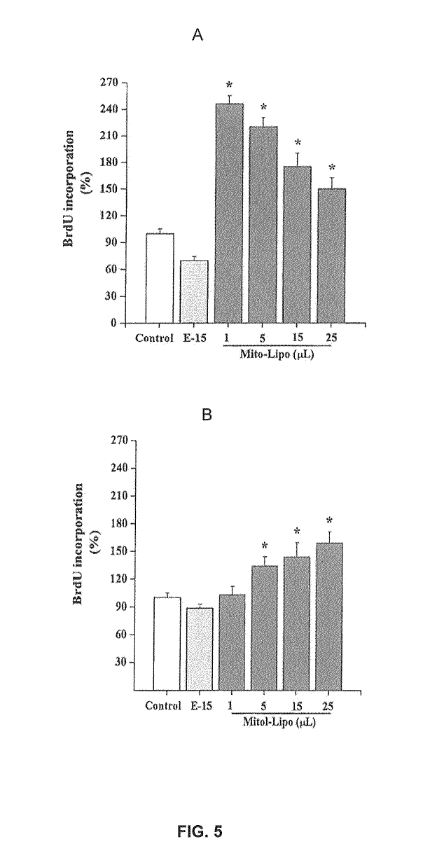

Mitochondrial transfer and proliferation of HT and NT-p° cells. BrdU incorporation in HT and NT-p° cells was measured after treatment with 1-, 5-, 15-, and 25 μl of the mitochondria-liposome complex. In HT cells, 5-25 μl of the complex caused a 2.5-fold increase in cell proliferation rates relative to untreated cells (N=3, P<0.05, FIG. 5A). Empty liposomes inhibited HT cell proliferation. In NT-p° cells, treatment with mitochondria-liposome complex caused a 1.5- to 1.7-fold increase in cell proliferation rates relative to untreated cells (N=3, P<0.05) after treatment with 1-, 5-, 15- and 25 μl the complex. Empty liposomes caused a subtle suppression of the ability of NT-p° cells to proliferate (FIG. 5B). Treatment of endothelial cells (HT) with the mitochondria liposome complex led to the recovery of HT cell paracrine function (Supplemental Materials) as shown by a 20% increase in nitric oxide released over 24 hours relative to untreated HT cells.

Integrity of mitochondrial structure and mitochondrial transfer. Isolated mitochondria were heat shocked (120 s, 95° C.), enclosed in liposomes, and delivered to HT cells. In control cells, ATP level was 7.6±0.6 nmol/mg protein; however, treatment with 1-, 5-, 15-, and 25 μl of the liposome complex formed with heat-inactivated mitochondria decreased total ATP to 3.4±0.5, 4.3±0.8, 2.9±0.3 and 2.3.4±0.4 nmol/mg protein, respectively (N=3, FIG. 6). In addition, if mitochondria were purposely ruptured by hypotonic swelling (30 min, 4° C.) and sonication (5, 30 s intervals), enclosed in liposomes, and delivered to recipient endothelial cells, the recipient HT cells died within 12 hours of the treatment (not shown).

The described embodiments are to be considered in all respects only as illustrative and not restrictive, and the scope of the present invention is not limited to the description. Those of skill in the art may recognize changes, substitutions, adaptations and other modifications that may nonetheless come within the scope of the present invention and range of the present invention.

### LIST OF REFERENCE CITED

Each of the following references is incorporated by reference in its entirety.

1. Ballinger S W. Mitochondrial dysfunction in cardiovascular disease. Free Radio Bid Med 2005:38;1278-95.

2. Gutierrez J, Ballinger S W, barley-Usmar V M, Landar A. Free radicals, mitochondria, and oxidized lipids: the emerging role in signal transduction in vascular cells. Ciro Res 2006;99:924-32.

3. Gutierrez J, Ballinger S W, Darley-Usmar Landar A. Free radicals, mitochondria, and oxidized lipids: the emerging role in signal transduction in vascular cells. Circ Res 2006;99:924-32.

4. Ballinger S W, Patterson C, Knight-Lozano C A, Burow D L, Conklin C A, Hu Z, et al. Mitochondrial integrity and function in atherogenesis. Circulation 2002;106:544-9.

5. Tuppen H A, Blakely E L, Turnbull D M, Taylor R W. Mitochondrial DNA mutations and human disease. Biochim Biophys Acta 2010;1797:113-28.

6. Wu V T, Wu S B, Lee W Y, Wei Y H. Mitochondrial respiratory dysfunction-elicited oxidative stress and posttranslational protein modification in mitochondrial diseases. Ann N Y Acad Sci 2010;1201:147-56.

7. Chouchani E T, Hurd T R, Nadtochiy S M, Brookes P S, Fearnley I M, Lilley K S, et at Identification of S-nitrosated mitochondrial proteins by S-nitrosothiol difference in gel electrophoresis (SNO-DIGE): implications for the regulation of mitochondrial function by reversible S-nitrosation, Biochem J 2010;430:49-59.

8. Gottlieb R A, Carreira R S. Autophagy in health and disease. 5. Mitophagy as a way of life, Am J Physiol Cell Physiol 2010;299:C203-C210.

9. Tachibana M, Sparman M, Sritanaudomchai H, Ma H, Clepper L, Woodward J, et al. Mitochondrial gene replacement in primate offspring and embryonic stem cells. Nature 2009;461:367-72.

10. Craven L, Tuppen H A, Greggains G D, Harbottle S J, Murphy J L, Cree L M, et al. Pronuclear transfer in human embryos to prevent transmission of mitochondrial DNA disease. Nature 2010;465:82-5.

11. King M P, Attardi C. Injection of mitochondria into human cells leads to a rapid replacement of the endogenous mitochondrial DNA. Cell 1988;52:811-9.

12. King M R Attardi G. Human cells lacking mtDNA: repopulation with exogenous mitochondria by complementation, Science 1989;246:500-3.

13. Weissig V, Boddapati S V, Cheng S M, D'Souza G G. Liposomes and liposome-like vesicles for drug and DNA delivery to mitochondria, J Liposome Res 2006;16:249-64.

14. Yamada Y, Akita H, Kogure K, Kamiya H, Harashima H. Mitochondrial drug delivery and mitochondrial disease therapy—an approach to liposome-based delivery targeted to mitochondria. Mitochondrion 2007;7:63-71.

15. Spees J L, Olson S D, Whitney M J, Prockop D J. Mitochondrial transfer between cells can rescue aerobic respiration. Proc Natl Aced Sci USA 2006;103:1283-8.

16. Csordas A. Mitochondrial transfer between eukaryotic animal ells and its physiologic role. Rejuvenation Res 2006;9:450-4.

17. Katrangi E, D'Souza G, Boddapati S V, Kulawiec M, Singh K K, Bigger B, et al. Xenogenic transfer of isolated murine mitochondria into human rho0 cells can improve respiratory function, Rejuvenation Res 2007;10:561-70.

18. King M P, Attardi G. Isolation of human cell lines lacking mitochondrial DNA. Methods Enzymol 1996;264:304-13.

19. Yang Y, Loscalzo J. S-nitrosoprotein formation and localization in endothelial cells. Proc Natl Acad Sci USA 2005;102:117-22.

20. Moran O, Sandri C, Panfili E, Stuhmer W, Sorgato M C. Electrophysiological characterization of contact sites in brain mitochondria. J Biol Chem 1990;265:908-13.

21. Criado M, Keller B U. A membrane fusion strategy for single-channel recordings of membranes usually non-accessible to patch-clamp pipette electrodes, FEBS Lett 1987;224;172-6.

22. Ballarina C, Sorgato M C. Patch-clamping of mitochondrial membranes and proteoliposomes. In: Brown G C, Cooper C E, eds. Bioenergetics, 1 ed. Oxford, England: Oxford University Press, 1995:133-157.

23. Scaduto R C, Jr., Grotyohann L W. Measurement of mitochondrial membrane potential using fluorescent rhodamine derivatives. Biophys J 1999;76:469-77.

24. Clark M A, Shay J W. Mitochondrial transformation of mammalian cells. Nature 1982;295:605-7.

25. Zhang Q, Raoof M, Chen Y, Sumi Y, Sursal T, Junger W, et al. Circulating mitochondrial DAMPs cause inflammatory responses to injury. Nature 2010;464:104-7.

26. Noshing A, Fujioka K, Oku T, Nakamura S, Suga M, Yamaguchi Y, et al, Quantum dots targeted to the assigned organelle in living cells, Microbiol Immunol 2004;48:985-94.

27. Sud N, Wells S M, Sharma S, Wiseman D A, Wilham J, Black S M. Asymmetric dimethylarginine inhibits HSP90 activity in pulmonary arterial endothelial cells: role of mitochondrial dysfunction. Am J Physiol Cell Physiol 2008;294:C1407-01418.

28. Moscho A, Orwar O, Chiu D, Modi B, Zare R. Rapid preparation of giant unilamellar vesicles Proc. Natl. Acad. Sci. 1996; 93:11443-11447.

29. Rawat M, Signh D, Saraf S., and Saraf S. Lipid Carriers: A Veratile Delivery Vehicle for Proteins and Peptides Yakugaku Zasshi 2008; 128:269-280.

1. A composition comprising a lipid carrier comprising at least one mitochondria.

2. The composition of claim 1, wherein the lipid carrier is selected from a liposome, a lipid microtubule, a lipid microbubble, and a lipid microsphere.

3. The composition of claim 1, wherein the liposome comprises a phospholipid bilayer comprising at least one type of phospholipid selected from L-α-phosphatidylcholine, dipalmitoleoyl L-α-phosphatidylcholine, β-arachidonoyl γ-palmitoyl L-α-phosphatidylcholine, phosphatidyl glycerol, phosphatidyl inositol, phosphatidylserine, phosphatidic acid, or cardiolipin phosphatidylglycerol, phosphatidylserine, sphingomyelin dicetylphosphate, phosphatidylethanolamine, cholesterol, and PEG-lipids.

4. The composition of claim 3, wherein the liposome is unilamellar.

5. The composition of claim 3, wherein the liposome is multilamellar

6. The composition of claims 1, wherein the diameter of the lipid carrier is at least 0.5 μm.

7. The composition of claim 6, wherein the diameter of the lipid carrier is at least 1.0 μm.

8. The composition of claims 1, wherein the lipid carrier further comprises a targeting moiety.

9. The composition of claim 8, wherein the targeting moiety is an antibody or a receptor ligand.

10. The composition of claim 1, wherein the mitochondria are obtained from a heterologous donor or an autologous source.

11. The composition of claim 10, wherein the mitochondria are obtained from mature umbilical cord cells or from umbilical cord stem cells.

12. The composition of claim 1, further comprising a pharmaceutically acceptable carrier.

13. A method of delivering exogenous mitochondria to a cell having reduced or defective mitochondria, comprising contacting the cell with the composition of claim 1 under conditions suitable for uptake of the mitochondria by the cell.

14. The method of claim 13, wherein the cell is deficient in mitochondria or comprises defective mitochondria.

15. The method of claim 14, wherein the cell is comprised within a mammal, and wherein contacting the cell is performed by administering the composition to the mammal.

16. The method of claim 15, wherein the composition is administered orally, intraperitoneally, intravenously, subcutaneously, intramuscularly, or by inhalation.

17. A cell comprising exogenous mitochondria made by the method of claim 13.

18. A method of treating and/or reversing progression of a disorder associated mitochondrial dysfunction in a mammalian subject in need thereof, comprising administering the composition of claim 1.

19. The method of claim 18, wherein the mammalian subject has one or more of a cardiovascular disease, a mitochondrial disease, and a mitochondrial disorder associated with aging.
